(19) United States
(12) Patent Application Publication
(10) Pub. No.: US 2023/0303424 A1
(43) Pub. Date: Sep. 28, 2023

(54) Biological glass fiber for regenerative medical materials and applications thereof

(71) Applicant: TAIWAN FIBER OPTICS, INC., Taipei City (TW)

(72) Inventors: Luke Lu, San Diego, CA (US); Thomas J. Loretz, Waxhaw, NC (US); Hsiao Sen Tseng, Taichung (TW); Yu-Chih Chiang, Taipei City (TW)

(21) Appl. No.: 17/696,898

(22) Filed: Mar. 17, 2022

(51) Int. Cl. C03C 13/00; A61L 27/54; A61L 27/38; C03C 3/087; A61L 27/10

(52) U.S. Cl. CPC C03C 3/087; A61L 27/10; A61L 27/3834; A61L 27/54; C03C 13/00; A61L 2300/414; A61L 2430/02; C03C 2213/02

(57) ABSTRACT

The present invention provides a biological glass fiber for regenerative medical materials, comprising a biological glass or a biologically-inert glass made of a glass chemical composition. The glass chemical composition comprises in weight percentage, 5 to 25 wt% Na₂O, 45 to 67 wt% SiO₂, 15 to 25 wt% CaO, 2 to 6 wt% P₂O₅, 1 to 8 wt% MgO, 8 to 12.5 wt% K₂O, 0.1 to 5 wt% total combined of non-toxic elements found in Group 5 and non-toxic elements found in Transition Metal Groups 3B, 4B and 5B, based on 100 wt% of the glass chemical composition; wherein the biological glass fiber forms a fiber configuration. The biological glass fiber is a soluble, resorbable, light transmittable and controllable absorption time regenerative material, and the biological glass fiber can mediate: therapeutic lymphangiogenesis, stem cell regeneration, bone cell regeneration and neurovascular regeneration, or it can be used as a cell carrier.

### (A) TECHNICAL FIELD OF THE INVENTION

The present invention relates to a biological glass fiber for regenerative medical materials and applications thereof. More particularly, it relates to the invention of biological glass fiber configurations made from known biologically-compatible glass fiber chemical compositions, which fiber configurations perform as chemically resorbable, light transmittable, mediated lymphangiogenesis materials, capable of inducing directional arrangement and proliferation of stem cells. Furthermore, the biological glass fiber has unique ability to mediate: stem cell regeneration, bone cell regeneration and neurovascular regeneration, or function as a cell carrier.

### (B) DESCRIPTION OF THE PRIOR ART

Regenerative medicine refers to the production of functional and vital organs and tissues to repair or replace unhealthy organs and tissues in the body due to aging, illness, or damage. Therefore, the field of regenerative medicine holds the promise of engineering damaged tissues and organs by stimulating the body’s own repair mechanisms to heal previously irreparable tissues or organs.

In 1969, Dr. Lawrence Hench developed a simple, four-component glass for use as a bone replacement material in animals, and more specifically, in humans. Eventually, Hench added additional components to this basic glass chemical composition and researchers applying this family of materials coined the name “Bioglass”. More recently, this material has been assigned to the class of materials known as “bioactive glasses,” (therefore, biologically-compatible glasses).

In recent years, human tissues and organs have been harvested in laboratories for transplantation into a patient’s body. Alternate methods include drug release in the body to stimulate the tissues in a patient to regenerate directly. New regenerative medical technologies have been developing rapidly in bone, cartilage, skin, bladder, ureter, kidney, liver and nerve sciences.

It is vital to choose regenerative materials, prudently, as cells are extremely sensitive to the physio-chemical properties of the culture environment. Mechanical properties and pore structures of materials used need consideration, as they affect tissue repair and growth after implantation. Regenerative materials may differentiate into adipocytes, chondrocytes, nerve cells, osteoblasts or muscle cells, depending upon culture conditions or the addition of specific growth factors.

In view of the prior art in this field, the authors of this invention have made an innovative breakthrough which overcomes the deficiencies of conventional delivery methods, and which improves the actual regenerative material chemistry efficacy. This invention applies novel approaches which leverage technologies developed for lighting and communication fiber optics manufacture to bring vastly improved regenerative medical materials to the market, promote the development of the industry and improve the health of society.

### SUMMARY OF THE INVENTION

The main purpose of the present invention is to provide a biological glass fiber and fiber configurations for regenerative medical materials and applications thereof. A single bare fiber configuration or a hybrid construction fiber configuration made from any of the biological glass fiber compositions described in this invention can generate cell activity mechanisms and offer environments in the body that cater to the cell and stimulate the integration of the immune system. Furthermore, since both dissolution and absorption of the fiber dictate functionality, the biological glass fiber of this invention is manufactured to precise structure size and unique formula ratio to adjust the dissolution and absorption rates. Furthermore, the biological glass fiber of this invention is a medical material which is used to mediate lymphangiogenesis and to induce the directional arrangement and proliferation of stem cells. Furthermore, with modification, variants have the potential to form a skeleton and nerve ducts.

To achieve the objectives above, the present invention makes use of a well-known, biological glass family of glass chemical compositions to fabricate fiber-based, regenerative medical materials, comprising a biological glass or a biologically-inert glass. The glass family composition of the biological glass comprises: 5 to 25 wt% Na₂O, 45 to 67 wt% SiO₂, 15 to 25 wt% CaO, and 2 to 6 wt% P₂O₅, based on 100 wt% of the glass chemical composition; wherein the biological glass fiber forms a single bare fiber configuration and a hybrid construction fiber configuration.

In some embodiments, the glass chemical composition of the biological glass further comprises 1 to 8 wt% MgO and 8 to 12.5 wt% K₂O.

In some embodiments, the glass chemical composition of the the biologically-inert glass comprises 0.1 to 5 wt% non-toxic elements in Group 5 and Transition Metal in Groups 3B, 4B and 5B, based on 100 wt% of the glass chemical composition.

Applying fabrication means adapted from the well-known art of biological glass fiber manufacture for communication and lighting technologies, the biological glass is made into either a single bare fiber or a hybrid construction fiber. These biological glass fibers can be solid core or capillary channel in nature. Furthermore, individual fibers may be gathered and combined to form more complex fiber-based constructions.

In some embodiments, the single bare fiber configuration is made of the biological glass or the biologically-inert glass from the family.

In some embodiments, hybrid-construction (henceforth, “hybrid”) fiber configuration beyond the single bare fiber configuration made of a biological glass may include: (1) a single clad fiber which for definition purposes is made of a biological core glass and a biologically-inert outer glass cladding, (2) a single bare fiber capillary made with biological glass, (3) a single bare fiber with multiple fused internal capillaries made from biological glass, (4) a plurality of single bare fibers made from biological glass in an unfused bundle construction, whose interstitial voids comprise natural biological glass capillaries, (5) a plurality of single bare fiber capillaries in an unfused bundle construction, whose interstitial voids comprise additional natural biological capillaries and (6) a plurality of single clad fibers in an unfused construction, whose interstitial voids comprise additional natural capillaries of biologically-inert glass.

In some embodiments, the hybrid construction fiber configuration comprises a single capillary channel fiber configuration made of the biological glass or the biologically-inert glass from the family.

In some embodiments, the hybrid construction fiber configuration comprises a hybrid solid fiber configuration made of two or more of the biological glasses, the biologically-inert glasses from the family or a combination thereof.

In some embodiments, the hybrid construction fiber configuration comprises a hybrid single capillary channel fiber configuration made of two or more of the biological glasses and biologically-inert glasses from the family or a combination thereof.

In some embodiments, the hybrid construction fiber configuration comprises a hybrid multiple capillary channel fiber configuration made of the biological glass or the biologically-inert glass from the family.

In some embodiments, the hybrid multiple capillary channel fiber configuration is made of two or more of the biological glass or the biologically-inert glass from the family and may have a circular or non-circular cross-sectional geometry.

In some embodiments, the single bare fiber configuration and the hybrid construction fiber configuration are made to include a bioinert glass with X-ray opacity. Furthermore, the glass chemical composition may contain specific biological additive materials to introduce X-ray opacity in one or more biological glass fibers, to create a new configuration, dependent upon application type. Moreover, the glass chemical composition may contain specific biologically-inert additive materials to introduce X-ray opacity in one or more biological glass fibers, to create a new configuration, dependent upon application type.

In some embodiments, the single bare fiber configuration and the hybrid construction fiber configuration can be made to have different regenerative material absorption times in a human or animal organism, based on either glass chemical composition modification or fiber component diameter sizing parameters.

In some embodiments, the single bare fiber configurations have diameters between 3 and 25 µm.

In some embodiments, the hybrid multiple capillary channel fiber configuration has circular diameters between 3 and 35 µm.

In some embodiments, the hybrid multiple capillary channel fiber configuration with non-circular cross-sectional geometries have apertures between 3 and 125 µm.

The main purpose of the present invention is to provide an application of biological glass fibers for regenerative medical materials formed from a glass chemical composition mentioned above, which are bio-engineered specifically for regenerative medical material devices: (1) providing therapeutic lymphangiogenesis, (2) inducing directional arrangement and proliferation of stem cells, (3) inducing stem cell regeneration, (4) inducing bone cell regeneration, (5) inducing neurovascular regeneration or (6) acting as cell carriers.

In some embodiments, the biological glass fiber configurations and application described above revive macrophages as lymphatic endothelial progenitors and promote draining of aqueous humor, without fibrosis, through therapeutic lymphangiogenesis.

In some embodiments, biological glass fiber can be manufactured to co-culture with dental pulp stem cells (DPSCs), causing the DPSCs to grow towards the biological glass fibers within 15 to 18 hours, to grow on the biological glass fibers within 22 to 25 hours and to form dentin-forming odontoblasts in days. In general, stem cells are defined as undifferentiated cells that exhibit self-renewal and multi-lineage differentiation ability. In addition to dentin-forming odontoblasts, DPSCs can differentiate into mesodermal and non-mesodermal tissue cells that include: osteoblasts, adipocytes, chondrocytes, myocytes, neuronal and endothelial cells, melanocytes, hepatocytes and retinal stem cell.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a schematic view of the present invention for a single bare fiber configuration.

FIG. 2A is a schematic view of the present invention for a hybrid construction fiber configuration.

FIG. 2B is an SEM image of Example-1 of the present invention for a hybrid solid fiber configuration.

FIG. 2C is an SEM image of Example-2 of the present invention for a hybrid solid fiber configuration.

FIG. 2D is an SEM image of Example-3 of the present invention for a hybrid solid fiber configuration.

FIG. 2E is an SEM image of Example-4 of the present invention for a hybrid solid fiber configuration.

FIG. 2F is an SEM image of Example-5 of the present invention for a hybrid solid fiber configuration.

FIG. 3A is a photomicrograph image of Example-1 of the present invention for a single capillary channel fiber configuration.

FIG. 3B is a photomicrograph image of Example-2 of the present invention for a single capillary channel fiber configuration.

FIG. 4A is a photomicrograph image of Example-1 of the present invention for a hybrid single capillary channel fiber configuration.

FIG. 4B is a photomicrograph image of Example-2 of the present invention for a hybrid single capillary channel fiber configuration.

FIG. 5A is a photomicrograph image of Example-1 of the present invention for the tip of a hybrid multiple capillary channel fiber configuration.

FIG. 5B is a photomicrograph image of Examples-2 through 12 of the present invention for a hybrid multiple capillary channel fiber configuration.

FIG. 5C is a photomicrograph image of Examples-3 of the present invention for a hybrid multiple capillary channel fiber configuration.

FIG. 6 is a photomicrograph image of a histopathological examination of cells introduced to biological glass fiber application types of the present invention, engineered with specific regenerative medical materials for the purpose of mediating lymphangiogenesis.

FIG. 7 is a photomicrograph image of a histopathological examination of cells introduced to biological glass fiber application types of the present invention, engineered with specific regenerative medical materials implanted hypodermically into tissue.

FIG. 8 shows photomicrograph image sequences of human Dental Pulp Stem Cells (DPSCs) growing on biological glass fiber application types of the present invention.

### DETAILED DESCRIPTION OF THE PREFERRED EMBODIMENTS

In the following paragraphs, a detailed description is provided for a thorough understanding of the Figures listed above. Well-known structures and devices will also be used schematically for better comprehension.

The term “biological” as used herein with “configurations” and “application” such as “biological glass fiber”, “biologically-inert glass fiber”, “hybrid biological glass fiber”, “biological glass fiber with X-ray opacity” and “bioinert glass fiber with X-ray opacity” means a biological material that can be applied to an organism. The term “compatible” means a biological material that can be absorbed by the organism. The term “bioinert” means a biological material that cannot be absorbed by the organism but does no harm to the organism.

### Preferred Embodiment 1

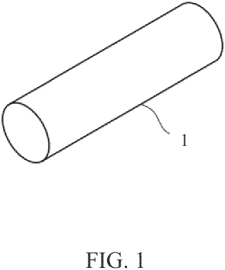

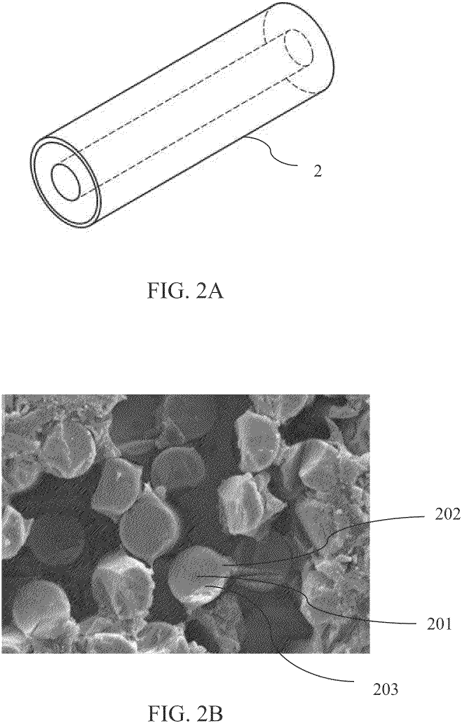

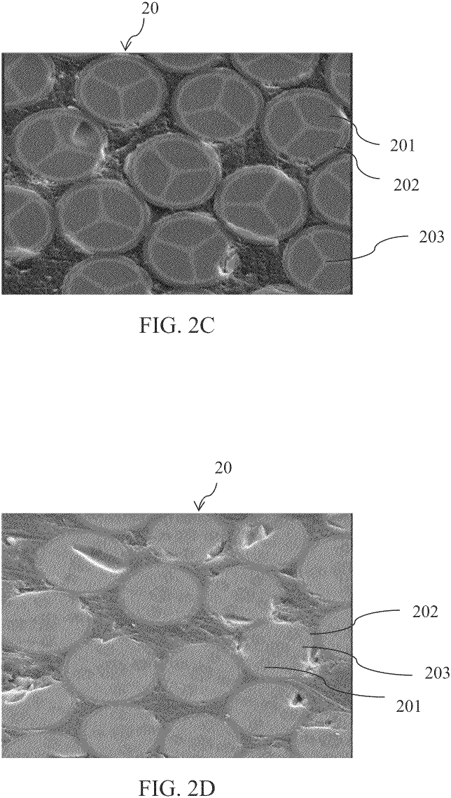

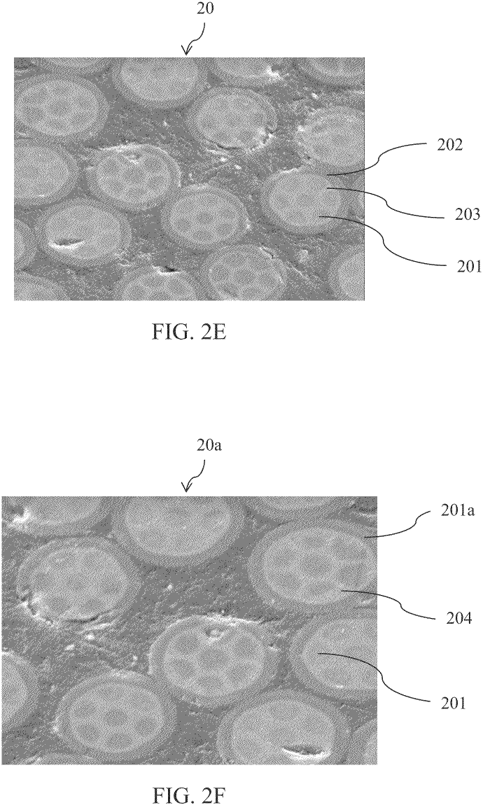

As shown in FIGS. 1 to 2F, the present invention provides a biological glass fiber for regenerative medical materials comprising a biological glass or a biologically-inert glass made of a glass chemical composition. The preferred glass chemical composition of the biological glass of the present invention is: 21.5 wt% Na₂O, 48.0 wt% SiO₂, 24.5 wt% CaO, and 6.0 wt% P₂O₅, based on 100 wt% of the glass chemical composition; wherein the glass chemical composition forms a biological glass fiber, and the biological glass fiber is made into a single bare fiber configuration or a hybrid construction fiber configuration, using known art. The regenerative chemical absorption time (henceforth, “absorption time”), which is the time that the regenerative glass material needs to integrate with biological tissue, is dependent upon the specific chemical composition of the glass.

As shown in FIG. 1, a single bare fiber configuration 1 is formed by the known art of melt-drawing and micro-fabrication technology; and as shown in FIG. 2A, the hybrid construction fiber configuration 2 with more than one layer is also formed by the melt-drawing and micro-fabrication technology. In other words, the hybrid construction fiber configuration 2 can be composed of two or more layers of biological glass material and the absorption time of each layer of the hybrid construction fiber configuration 2 can be tailored to meet the application type.

Alternatively, as shown in FIGS. 2B to 5C, there are multiple biological glass compositions incorporated into the hybrid solid fiber configuration. The shape of the biological glass fiber may be varied, and the absorption time of each biological glass fiber may be varied to meet the application type. Furthermore, the hybrid construction glass fiber configuration 2 is a basic building component and is incorporated and fused to form a hybrid solid fiber configuration 20, or a hybrid solid fiber configuration 20a, or a hybrid single capillary channel fiber configuration 21, or a hybrid multiple capillary channel fiber configuration 22 based on application type. Furthermore, the single bare fiber configuration 1 has a diameter between 3 and 25 µm; the hybrid solid fiber configuration 20 and hybrid solid fiber configuration 20a have diameters between 3 and 35 µm; and the hybrid single capillary channel fiber configuration 21 and the hybrid multiple capillary channel fiber configuration 22 have apertures between 3 and 125 µm, respectively.

The glass chemical compositions of the biological glass for regenerative medical materials form the biological glass fibers which can be used to provide therapeutic lymphangiogenesis, induce directional arrangement and proliferation of stem cells, induce stem cell regeneration, induce bone cell regeneration, induce neurovascular regeneration or function as cell carriers.

The regenerative medical glass materials of the present invention are different from conventional medical polymer materials, which depend on degradation and dissociation into tiny sharp particles prior to slow metabolization. The medical glass materials of the present invention dissolve just like sugar in water, quickly and completely. Moreover, the regenerative medical materials of the present invention can be absorbed by the human body safely and effectively. With the present invention, no foreign matter can be retained in the body and the undesirable phenomenon of biological tissue coating inherent with other methods does not occur. The patient does not experience the discomfort of sequelae, a pathological complication, brought on as the result of treatment, and the original implant site can be re-treated indefinitely, if necessary.

### Preferred Embodiment 2

The biological glass composition for regenerative medical materials comprises, in weight percentage, 6.5 wt% Na₂O, 50 wt% SiO₂, 20 wt% CaO, 6 wt% P₂O₅, 6.5 wt% MgO and 11 wt% K₂O, based on 100 wt% of the composition; wherein the composition of the biological glass is made into a single bare fiber configuration 1 or the hybrid-construction glass fiber configuration 2 by the known art of melt-drawing and micro-fabrication technology.

Furthermore, the hybrid construction fiber configuration 2 is a basic building component and is incorporated and fused to form a hybrid solid fiber configuration 20, or a hybrid solid fiber configuration 20a, or a hybrid single capillary channel fiber configuration 21, or a hybrid multiple capillary channel fiber configuration 22 based on application type. Furthermore, the single bare fiber configuration 1 has a diameter between 3 and 25 µm; the hybrid solid fiber configuration 20 and hybrid solid fiber configuration 20a have diameters between 3 and 35um; and the hybrid single capillary channel fiber configuration 21 and the hybrid multiple capillary channel fiber configuration 22 have apertures between 3 and 125 µm, respectively.

The chemical compositions of biological glass for regenerative medical materials form the biological glass fibers which can be used to provide therapeutic lymphangiogenesis, induce directional arrangement and proliferation of stem cells, induce stem cell regeneration, induce bone cell regeneration, induce neurovascular regeneration or function as cell carriers.

As shown in FIGS. 2B to 2F, the hybrid solid fiber configuration 20 comprises at least one biological glass fiber 201, a bioinert glass fiber 202, a bioinert glass fiber with X-ray opacity 203 or a biological glass fiber with X-ray opacity 204. In keeping with known art for biological glass fiber manufacture, a number of the biological glasses are surrounded by glasses with different refractive indices, softening temperatures and expansion coefficients, so that a circumferential surface around the plurality of the biological glasses is cladded and fused to form the hybrid solid fiber configuration 20. The hybrid solid fiber configuration 20 can induce bone cell regeneration, induce bone tissue osseointegration and osteoblast integration.

As shown in FIGS. 2B to 2F, the outer layer of the hybrid solid fiber configuration 20 is composed of the bioinert glass fiber 202, which is resistant to acid and alkali and is an insoluble material. The bioinert glass fiber 202 comprises traces of elements from the transition family of the Periodic Table and Group 5′s non-toxic elements. The transition elements include 3B group lanthanides, as well as 4B group and 5B group elements. In FIG. 2B, the outer layer of the hybrid solid fiber configuration 20 is composed of bioinert glass fiber 202; the center of the hybrid solid fiber configuration 20 is composed of biological glass fiber 201, which has good light transmittance; and between the outer (classing) layer and the center of the hybrid solid fiber configuration 20 is a layer of bioinert glass fiber with X-ray 203.

In FIG. 2C, there are 3 fan-shaped biological glass fiber 201 components in the hybrid solid fiber configuration 20 and the remainder are bioinert glass fiber with X-ray 203, which contain biologically-inert X-ray opacity additives and can be observed with X-ray cameras. In FIG. 2D, there are 5 biological glass fibers 201 in the hybrid solid fiber configuration 20. In FIG. 2E, there are 7 biological glass fibers 201 in the hybrid solid fiber configuration 20. The hybrid solid fiber configuration 20 is absorbed to form grooves and can be used for osseointegrating bone; and the rest are bioinert glass fiber 203 with X-ray opacity that can be observed by X-ray cameras.

In FIGS. 2B to 2E, the hybrid solid fiber configuration 20 is composed of biological glass fiber 201, bioinert glass fiber 202 and bioinert glass fiber with X-ray opacity 203, all with different dissolution times. As shown in FIG. 2F, the hybrid solid fiber configuration 20a is the same as that of the hybrid solid fiber configuration 20 of FIG. 2E, but the hybrid solid fiber configuration 20a of FIG. 2F is composed of biological glass fiber with X-ray opacity 204. In FIG. 2F, biological glass fibers 201a are composed of a chemical composition with enhanced dissolution rate and can be absorbed by the organism in about 2 \~7 days; the biological glass fiber with X-ray opacity 204 can be absorbed by the organism in about 30 days; and the biological glass fibers 201 can be absorbed by the organism in about 65 days.

Biological glass fibers 201 mediate therapeutic lymphangiogenesis. The purpose of hybrid solid fiber configuration 20 with X-ray opacity is to facilitate the interpretation of radiography by identifying the position of biological glass fibers 201 implanted in the organism. Furthermore, the hybrid solid fiber configuration 20 in FIGS. 2B, 2C and 2D may be composed of biological glass fibers with different dissolution rates, per application type.

It can be seen from Table-1 below that for all other conditions comparable (temperature, pH, liquid media, fiber diameter, etcetera), higher SiO₂ (silicon dioxide) content results in lower dissolution rates, which leads to longer regenerative material absorption times. The diameters of the biological materials in Table-1 are all 15 µm.

TABLE-1

Base Glass chemical composition Dissolution Rates

| Example | Na₂O (wt%) | SiO₂ (wt%) | CaO (wt%) | P₂O₅ (wt%) | Dissolution time (davs) |
|---|---|---|---|---|---|
| 1 | 18 to 25 | 45 | 25 | 2 to 6 | 7 |
| 2 | 18 to 24 | 50 | 23 | 2 to 5 | 30 |
| 3 | 5 to 15 | 60 | 20 | 2 to 4 | 65 |

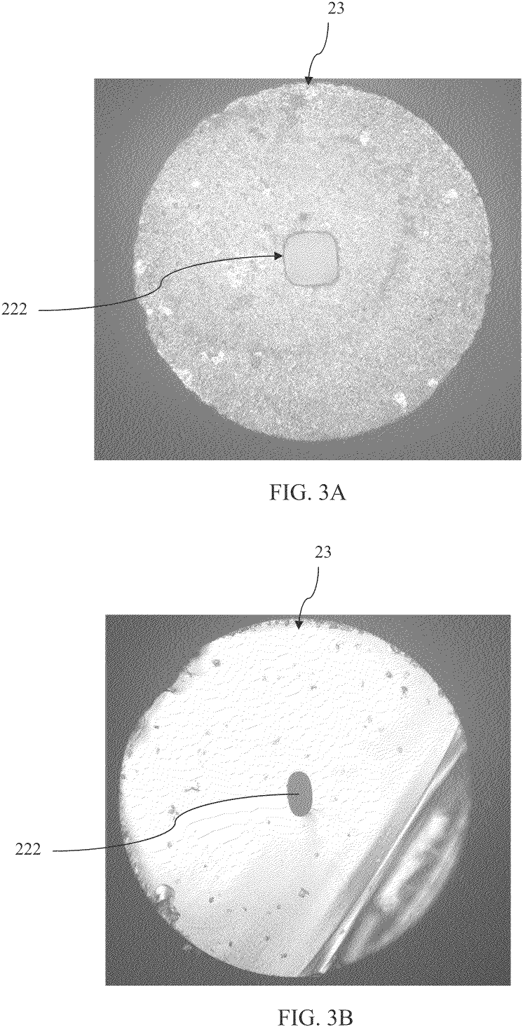

Referring to FIGS. 3A to 3B, FIG. 3A is an SEM image of the present invention for a single capillary channel fiber configuration 23 with a hybrid core 222 in a square geometry. FIG. 3B is an SEM image of the present invention for the single capillary channel fiber configuration 23 with the hybrid core 222 in an oval geometry.

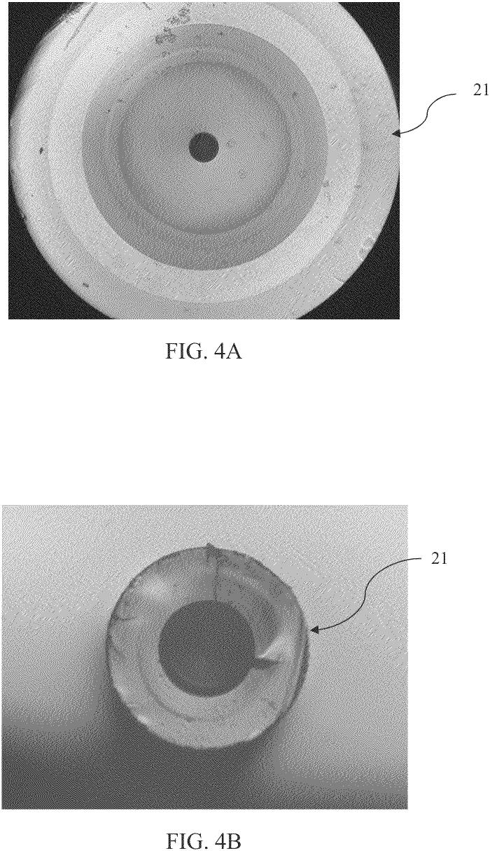

Referring to FIGS. 4A to 4B, FIG. 4A is an SEM image of the present invention for a hybrid single capillary channel fiber configuration 21. It demonstrates the extensive hybrid construction flexibility of the present invention for applying diverse types of biological and bioinert glass materials in sequential layers. FIG. 4B is an SEM image of the present invention for a 2-layer hybrid single capillary channel fiber configuration.

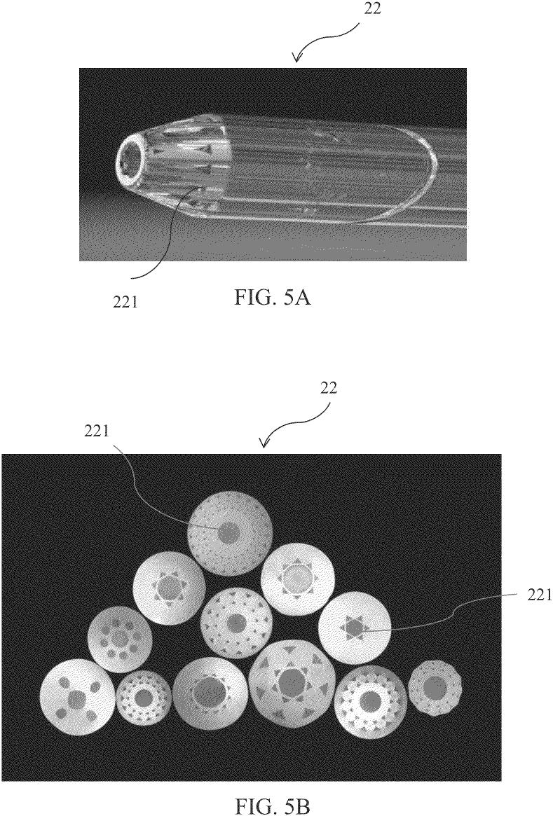

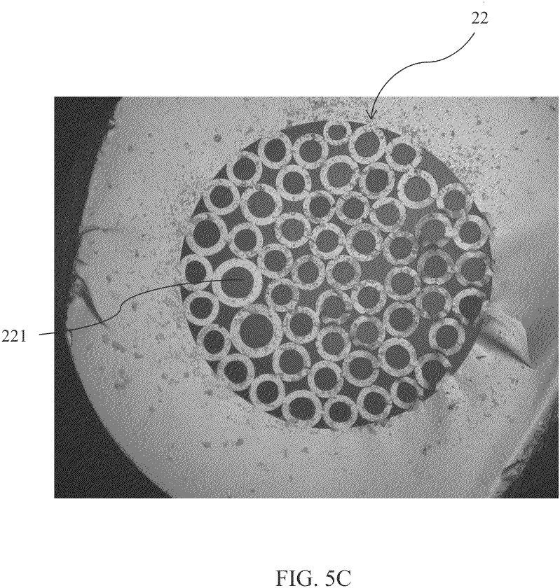

FIG. 5A is a photomicrograph image of the present invention for the tip of a hybrid multiple capillary channel fiber configuration.

FIG. 5B contains SEM images of the present invention for examples of conceivable cross-sections for hybrid multiple capillary channel fiber configuration and demonstrates the extensive flexibility of the hybrid construction method of manufacture. Any of the examples can be used as a drug carrier and chemical drugs can be carried in the channels. During treatment, a chemical drug required by a patient is placed in the channels and the hybrid fiber is placed in the human or animal body. When the chemical drug reaches the site to be treated, the chemical drug is released to the site for treatment. Furthermore, the number, shapes and sizes of the capillary holes may be easily varied to accommodate drug release rates, based on the application’s requirements. Furthermore, as shown, the capillary holes may be circles, or polygons and the present invention is not limited thereto.

FIG. 5C is an SEM image of the present invention for an example of a cross-section of a hybrid multiple capillary channel fiber configuration; wherein the hybrid multiple capillary channel fiber configuration 22 as the primary outer cladding has a plurality of fused channels made of capillary fiber 221.

### Lymphangiogenesis Test

### Experimental Animals

An 8-week-old male New Zealand white rabbit is used in the experiment, weighing about 2000 \~-2500g. All experimental animals are kept in an independent air-conditioned animal room, with room temperature maintained at 22° C. and relative humidity maintained at 45%, and water and feed are adequately supplied. Before the experiment, the experimental animals are given a period of at least 4 weeks to adapt to the environment. The breeding environment, handling and all experimental procedures of the experimental animals are in compliance with Guide for the Care and Use of Laboratory Animals of the National Institutes of Health (NIH).

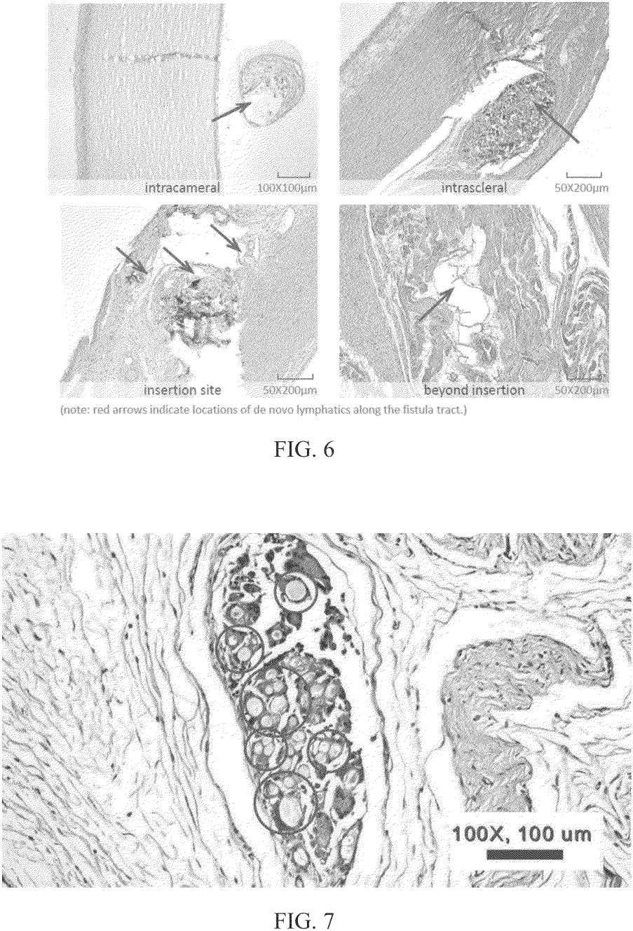

The area around the eyes of the New Zealand white rabbits is shaved, and then disinfected with iodine and 70% alcohol. The 26 or 27 gauge needle with the single bare fiber or the single constructed fiber implant inside the fiber bundle is inserted the sclera into the anterior chamber. The position of the single bare fiber or the hybrid construction fiber implant inside the fiber bundle is kept, and the 26 or 27 gauge needle is withdrawn and then the fiber bundle implant can be left to complete the operation. Please refer to FIG. 6,

FIG. 6 is a photomicrograph image of a histopathological examination of cells introduced to biological glass fiber application types of the present invention, engineered with specific regenerative medical materials for the purpose of mediating lymphangiogenesis. As shown in FIG. 6, red arrows indicate locations of de novo lymphatics along fistula tract. The biological glass fiber revives macrophages as lymphatic endothelial progenitors, and promote the lymphangiogenesis and draining of aqueous humor without fibrosis through therapeutic lymphangiogensis. After the lymphatic stent is placed in the eye, the lymphatic stent starts to drain and completely absorbed after three months, so that lymphangiogenesis immune system grows lymphatic vessels. After 7 months, the structure of the lymphatic vessels will spread from the thick tube to thinner, and the drainage pressure will be balanced naturally. After 7 months, the lymphatic vessels will still drain smoothly.

The back of the New Zealand white rabbits is shaved and disinfected with iodine and 70% alcohol. A small opening on the back of the New Zealand white rabbits is cut by a surgical knife and the biologically compatible glass fibers are implanted into the subcutaneous position of New Zealand white rabbits.

Table 2 below shows the hybrid construction fiber configuration implanted into the subcutaneous position of New Zealand white rabbits. After 12 weeks, the formation of the hybrid construction fiber configuration with different diameters and lymphatic vessels was observed.

TABLE 2

| | diameter (µm) | part of implantation after 12 weeks |
|---|---|---|
| Example a | 2 | mild inflammation and bleeding |
| Example b | 5 | mild inflammation and bleeding |
| Example c | 10 | no obvious inflammation, cysts, or bleeding |
| Example d | 15 | no obvious inflammation, cysts, or bleeding |
| Example e | 20 | no obvious inflammation, cysts, or bleeding |
| Example f | 25 | mild bleeding |

Table 3 below shows the hybrid multiple capillary channel fiber configuration implanted into the subcutaneous position of New Zealand white rabbits. After 12 weeks, the formation of the hybrid multiple capillary channel fiber configuration with different distances among channels and lymphatic vessels was observed. A channel distance refers to the distance between the two circumferences of the hybrid multiple capillary channel fiber configuration.

TABLE 3

| | channel distance (µm) | part of implantation after 12 weeks |
|---|---|---|
| Example 1 | 2 | mild inflammation and bleeding |
| Example 2 | 5 | mild bleeding |
| Example 3 | 10 | no obvious inflammation, cysts, or bleeding |
| Example 4 | 15 | no obvious inflammation, cysts, or bleeding |
| Example 5 | 20 | no obvious inflammation, cysts, or bleeding |
| Example 6 | 25 | mild bleeding |
| Example 7 | 30 | mild bleeding and cysts |
| Example 8 | 35 | mild bleeding and cysts |

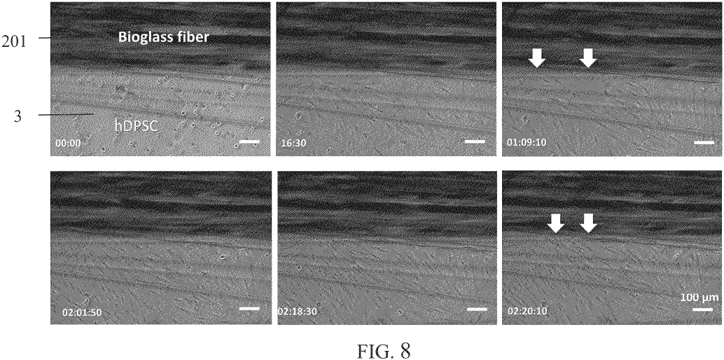

From the results of tables 2 to 3, the histopathological report of the 12-week rabbit subcutaneous implantation study. Simultaneously, refer to FIG. 7, the red circle represents cross-section of the hybrid multiple capillary channel fiber configuration. Moreover, the diameter of the hybrid multiple capillary channel fiber configuration is under appropriate conditions, the biological glass fibers revive macrophages as lymphatic endothelial progenitors, and promote the healing of therapeutic lymphangiogenesis. The subcutaneous implantation site does not show obvious inflammation, cysts, hemorrhage, necrosis or signs of discoloration.

Please refer to FIG. 8, FIG. 8 is a SEM image of human dental pulp stem cells growing on the biologically compatible glass fiber of the present invention.

As shown in FIG. 8, the biologically compatible glass fibers have directionality. When human dental pulp stem cells (hDPSC) 3 is put into the single bare fiber configuration or the hybrid construction fiber configuration, the human dental pulp stem cells 3 will grow along the biological glass fibers 201; wherein the lower left corner of each photo in FIG. 8 is the time of growing the human dental pulp stem cell. When the biological glass fibers 201 co-culture with the human dental pulp stem cells 3, the human dental pulp stem cells 3 have tended to the biological glass fibers 201 after 16 hours. Further, the human dental pulp stem cells 3 have tended to the biological glass fibers 201 after 18 hours; the human dental pulp stem cells 3 have grown on the biological glass fibers 201 after 22 hours; and the human dental pulp stem cells 3 will grow on the biological glass fibers 201 after 24 hours. The situation is more obvious that a large number of human dental pulp stem cells 3 grow close to the biological glass fibers 201 after 48 hours.

The application of other biological glass chemical compositions suitable for regenerative medical materials, which either have already been developed or may be developed in the future, into fiber configurations of any type described mentioned for purposes of introducing said regenerative medical materials into living organisms.

The above descriptions for each figure explain the principles of the disclosure and its practical applications. The embodiments depicted above and the appended drawings are exemplary and are not intended to be exhaustive or to limit the scope of the disclosure. Modifications and variations are possible in view of the above teachings.

I claim:

1. A biological glass fiber for regenerative medical materials, comprising a biological glass or a biologically-inert glass made of a glass chemical composition, the glass chemical composition of the biological glass comprising: 5 to 25 wt% Na₂O, 45 to 67 wt% SiO₂, 15 to 25 wt% CaO, and 2 to 6 wt% P₂O₅, based on 100 wt% of the glass chemical composition; wherein the biological glass fiber forms a single bare fiber configuration and a hybrid construction fiber configuration.

2. The biological glass fiber mentioned in claim 1, wherein the glass chemical composition of the biological glass further comprises 1 to 8 wt% MgO, 8 to 12.5 wt% K₂O.

3. The biological glass fiber mentioned in claim 1, wherein the glass chemical composition of the biologically-inert glass comprises 0.1 to 5 wt% non-toxic elements in Group 5 and Transition Metal in Groups 3B, 4B and 5B, based on 100 wt% of the glass chemical composition.

4. The biological glass fiber mentioned in claim 1, wherein the single bare fiber configuration is made of the biological glass or the biologically-inert glass from the family.

5. The biological glass fiber mentioned in claim 1, wherein the hybrid construction fiber configuration comprises a single capillary channel fiber configuration made of the biological glass or the biologically-inert glass from the family.

6. The biological glass fiber mentioned in claim 1, wherein the hybrid construction fiber configuration further comprises a hybrid multiple capillary channel fiber configuration made of the biological glass or the biologically-inert glass from the family.

7. The biological glass fiber mentioned in claim 1, wherein the hybrid construction fiber configuration further comprises a hybrid solid fiber configuration made of two or more of the biological glasses, the biologically-inert glasses from the family or a combination thereof.

8. The biological glass fiber mentioned in claim 1, wherein the hybrid construction fiber configuration comprises a hybrid single capillary channel fiber configuration made of two or more of the biological glasses, biologically-inert glasses from the family or a combination thereof.

9. The biological glass fiber mentioned in claim 6, wherein the hybrid multiple capillary channel fiber configuration is made of two or more of the biological glass or the biologically-inert glass from the family and may have a circular or non-circular cross-sectional geometry.

10. The biological glass fiber mentioned in claim 1, wherein the single bare fiber configuration and the hybrid construction fiber configuration are made to include a bioinert glass with X-ray opacity.

11. The biological glass fiber mentioned in claim 1, wherein the single bare fiber configuration and the hybrid construction fiber configuration can be made to have different regenerative material absorption times in a human or animal organism, based on either glass chemical composition modification or fiber component diameter sizing parameters.

12. The biological glass fiber mentioned in claim 1, wherein the single bare fiber configurations have diameters between 3 and 25 µm.

13. The biological glass fiber mentioned in claim 9, wherein the hybrid multiple capillary channel fiber configuration has circular diameters between 3 and 35 µm.

14. The biological glass fiber mentioned in claim 9, wherein the hybrid multiple capillary channel fiber configuration with non-circular cross-sectional geometries have apertures between 3 and 125 µm.

15. An application of biological glass fibers for regenerative medical materials formed from a glass chemical composition mentioned in claim 1, inducing therapeutic lymphangiogenesis, or inducing directional arrangement and proliferation of stem cells, or stem cell regeneration, or inducing bone cell regeneration, or inducing neurovascular regeneration or acting as a cell carrier.

16. The application mentioned in claim 15, wherein macrophages are revived as lymphatic endothelial progenitors and promotes draining of aqueous humor without fibrosis through therapeutic lymphangiogenesis.

17. The application mentioned in claim 15, wherein the biological glass fibers co-culture with dental pulp stem cells (DPSCs), causing the DPSCs to grow towards the biological glass fibers, with the end result being that the DPSCs grow on the biological glass fibers and integrate to dentin-forming odontoblasts in a clinically useful period of time.
